(19) United States
(12) Patent Application Publication
(10) Pub. No.: US 2016/0328929 A1
(43) Pub. Date: Nov. 10, 2016

(54) CEMENTITIOUS MATERIAL STRUCTURE WITH SENSORS, MANUFACTURING METHOD AND OPERATION METHOD THEREOF

(71) Applicants: SECIL - COMPANHIA GERAL DE CAL E CIMENTO, S.A., Lisboa (PT); CENTI-CENTRO DE NANOTECNOLOGIA E DE MATERIAIS TÉCNICOS FUNCIONAIS E INTELIGENTES, Vila Nova Famalicao (PT)

(72) Inventors: Angela Maria JESUS DE SEQUEIRA SERRA NUNES, Vila Nogueira de Azeitao (PT); Vitor Vermelhudo, Azeitao (PT); Joäo Manuel Carvalho Gomes, Ruilhe (PT); Ricardo Daniel Marques Pessoa, Vila Nova de Gaia (PT); Vasco Gonçalves Pimenta Machado, Tamel (Sao Verissimo) (PT); Joana Da Fonseca E Branquinho de Pais Monteiro, Nelas (PT); José Joaquim Poças Gonçalves, Braga (PT); Bruna Gabriela Silvestre Mendes Pinto de Moura, Vila Nova De Famalicao (PT); Jaime Rafael Pinto Lopes, Guifoes (PT); Joana Diniz Da Fonseca, Maia (PT)

(21) Appl. No.: 15/149,133

(22) Filed: May 8, 2016

(30) Foreign Application Priority Data
May 7, 2015 (PT) 108448

(51) Int. Cl. G08B 5/36; A61B 5/024; G08B 3/10

(52) U.S. Cl. CPC G08B 5/36; G08B 3/10; A61B 5/02405; A61B 5/02416

(57) ABSTRACT

The present invention relates to the field of construction, involving knowledge of building materials and sensors technology.

The invention consists of a cementitious material structure with sensors, which includes plurality of sensing elements (1) integrated on at least one substrate (2), and wherein the set of the plurality of sensing elements (1) and the at least one substrate (2) is involved in cementitious material. The invention thus enables a wide variety of ways to interact with construction structures, while allowing durability and robustness of these structures and their sensing elements (1).

The present invention also comprises a manufacturing method of a cementitious material structure with sensors, which includes the integration of sensing elements (1) on at least one substrate (2), its insertion into a formwork and filling with cementitious material.

### FIELD OF THE INVENTION

The present invention relates to the field of construction, involving knowledge of building materials and sensors technologies. More specifically, it involves cementitious materials and waterproofing materials, among others, as well as different technologies of contact or proximity sensors. The intersection of these technologic fields allows achieving constructive structures with different shapes, functions and dimensions, which interact with external elements.

Said cementitious materials are suitable for any kind of constructions, such as construction or rehabilitation, either inside or outside, of buildings, roads, street furniture, among others.

The object of the invention also relates to the development, of sensors suitable for interacting with human users.

Thus, the present invention aims to introduce interactivity in cementitious material structures, through the integration of sensors during the concreting process. These structures have application in the construction/rehabilitation of buildings, in highways, street furniture, hospital facilities, among others.

### BACKGROUND OF THE INVENTION

The closest antecedents of the present invention are found in the internal parameter monitoring systems of constructive structures, for continuous monitoring purposes and, therefore, predictive maintenance.

Such systems are intended to monitor the response of structures over time, either in the construction phase itself or during its lifetime, by placing sensors on the surface of the cementitious structures, by drilling the structure and by placing the sensors on the inside, or by placing the sensors inside during its construction.

The latter type of systems is the one that matters for purposes of the present invention, in which sensors are embedded into the structure during its construction and become part of the structure itself.

Patent application publication No. WO 2007025172A2 discloses a system for monitoring the healing process of concrete, in which devices with sensing and wireless communication capabilities are placed inside the concrete during concreting, allowing to monitor the temperature at different points inside the structure, while concrete is drying.

Patent application publication No. CN 102255959A discloses a system in which devices with strain gauges and temperature sensors and having wireless communication capability are embedded in a concrete structure during concreting, so that they monitor parameters which allow to know the conservation state of the structure throughout its lifetime.

### Technical Problems Solved

In the present invention, it was intended to monitor external parameters and not internal structure parameters. With this change, it is intended to obtain interaction with the outside of the structure, typically in solutions of interactivity with a human user. Thus, the configuration, the arrangement, the sensor type and other features are distinct from those known in prior art, in a way that sensors embedded into the cementitious structures can detect variables external to the structure.

On the other hand, it is necessary to carry out a process different from those known in prior art to obtain an encapsulation, and therefore durability and reliability such that allow a proper operation of the sensors set over time, including providing resistance to the concreting process of the cementitious material structures.

Additionally, with the present invention it is intended to provide contactless user interfaces by making use of porosity characteristics of the cementitious materials.

### SUMMARY OF THE INVENTION

It is therefore the object of the present invention a structure of cementitious material with sensors, which includes at least one substrate (2), a plurality of sensing elements (1) placed on the at least one substrate (2) and cementitious material involving the whole set.

Such a configuration is related to the sensing characteristics of the cementitious material structure. It is intended to measure a certain physical variable along the surface of the cementitious material structure, or part of it, thus defining an area over which the variable is detected, resulting in an adequate sensitive surface for monitoring a certain variable over the outer surface (4) of the structure.

More specifically, the cementitious material structure with sensors further includes a waterproofing material layer (3) covering the set of the plurality of sensing elements (1) and at least one substrate (2).

The waterproofing material layer (3) allows the sensing elements (1) and the substrate (2), which typically are barely suitable to be involved with cementitious material, to be involved by thus allowing an external finishing only in cementitious materials while maintaining the robustness and reliability characteristics of the set of sensors. The waterproofing material layer (3) is therefore placed between the set of the plurality of sensing elements (1) together with the at least one substrate (2) and the cementitious material.

In this configuration, the plurality of sensing elements (1) is printed on the substrate (2). To make possible this configuration, the sensing elements (1) are printed on the substrate (2) by means of different possible techniques.

Said sensitive surface is also involved in a waterproofing material, which encapsulates it in order to withstand the concreting process and throughout the lifetime of the structure, and involved in cementitious material, which provides a finishing that hides the plurality of sensing elements (1) and that corresponds to the outer surface (4) of the structure.

It is also an object of the present invention a manufacturing method of the cementitious material structure with sensors, which includes the following steps:

a) integrating a plurality of sensing elements (1) on at least one substrate (2);

b) inserting the encapsulated set of the plurality of sensing elements (1) and the at least one substrate (2) inside the formwork;

c) filling with cementitious material.

Through the method of the present invention, it is possible to obtain a cementitious material structure with sensors, which complies with said capability requirements for monitoring variables on the surface of the structure, and having a suitable encapsulation for the durability and reliability of the sensing elements (1).

This method may further include, for obtaining some of the configurations, the following step performed between steps a) and b):
encapsulation of the set of the plurality of sensing elements (1) and the at least one substrate (2) with a waterproofing material (3).

### DESCRIPTION OF THE FIGURES

FIG. 1—representation of a cementitious material structure with sensors according to the present invention, showing a plurality of sensing elements (1), a substrate (2) on which they are integrated—printed in the case of the figure—and a base of cementitious material on which the substrate (2) is placed. It is also represented the cementitious material which covers and fills this set (slab shown at left) as well as one sensing element extension (11) for connection to the other electronic elements with functions such as signal conditioning or signal processing.

FIG. 2—another representation of a cementitious material structure with sensors according to the present invention, showing a plurality of sensing elements (1), a substrate (2) on which they are printed, and a base of cementitious material on which the substrate (2) is placed.

FIG. 3—representation of a set of substrate (2) with a plurality of sensing elements (1) which define a base assembly (14) (these elements are not shown)—covered by cementitious material and placed in a mold for filling with cementitious materials (13). It is also represented a connection cable to power means (12).

FIG. 4—representation of the mesh of sensing elements (1) printed on a substrate (2), which allows to map the area where it is held the touch and/or where a user approaches to the structure of cementitious material with sensors. This action activates the closest sensing element (1), initiating an activation process of at least one actuator element (10) or remote communications element. Four peripheral sensing elements (6) and a central sensing element (5) are visible.

FIG. 5—representation of the manufacturing method of the present invention. The step of encapsulation with waterproofing material (3) is shown as optional, since it does not make part of all the possibilities of the method of the present invention and it is only suitable for setting up some configurations of the cementitious material structure with sensors of the present invention.

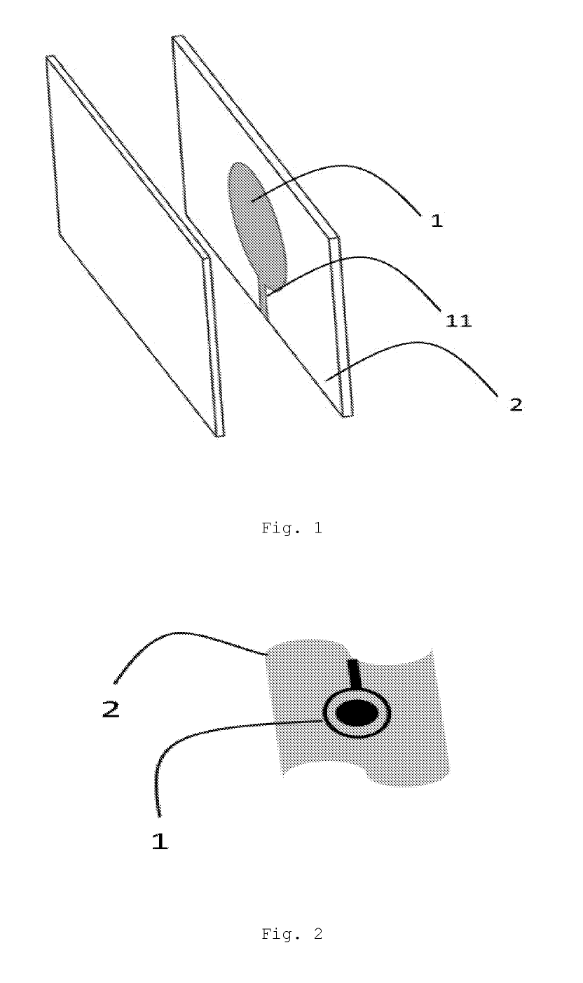

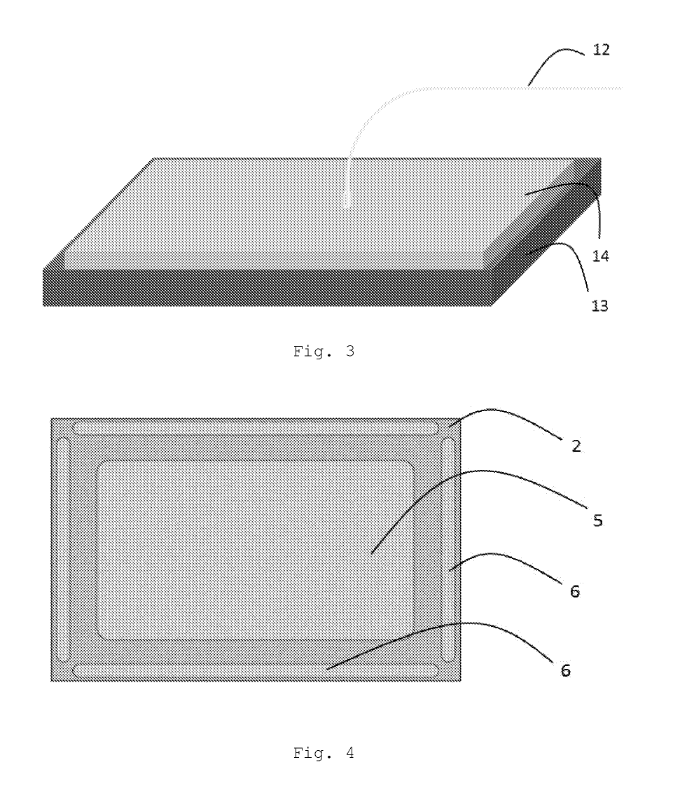

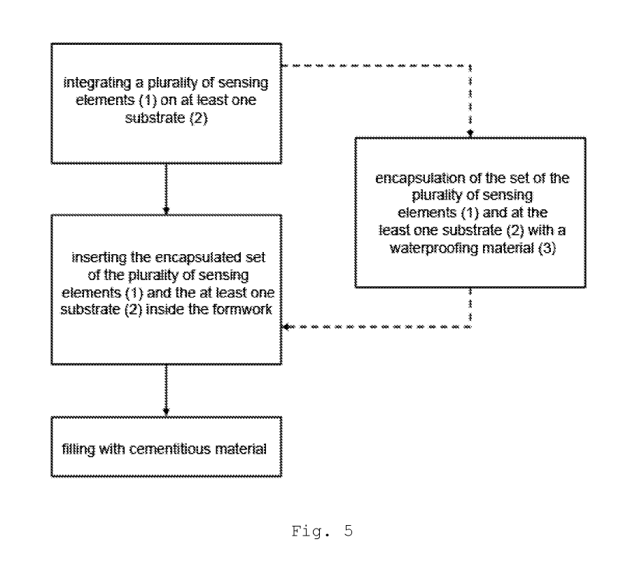

### DETAILED DESCRIPTION OF THE INVENTION

The present invention allows any cementitious material structure, for instance a concrete wall, becomes interactive with the outside, specifically incorporating a plurality of sensing elements (1) therein.

As mentioned before, systems are known that enable the incorporation of sensors into cementitious material structures, being however suitable for monitoring variables related to the structure condition.

Thus, the present invention comprises a configuration different from those of prior art and includes a cementitious material structure with sensors comprising a plurality of sensing elements (1) arranged on at least one substrate (2), and wherein the set of the plurality of sensing elements (1) with the at least one substrate (2) is involved in cementitious material.

The set of the plurality of sensing elements (1) with the at least one substrate (2) included in the structure is covered by a waterproofing material layer (3) which separates it from the involving cementitious material. This waterproofing material layer (3) allows said set to be encapsulated so that it resists the concreting process and remains in operation during the lifetime of the structure. The set of the plurality of sensing elements (1), with the at least one substrate (2) and with the involving cementitious materials is called base assembly (14).

Additionally, this structure has a configuration in which the plurality of sensing elements (1) is arranged on a surface substantially parallel to an outer surface (4) of the cementitious material structure with sensors.

Such a configuration enables the alignment of the contact or outer interaction surface with the surface with sensors, thereby achieving the measurement of the variable along a particular area with sensors.

In one embodiment of the present invention, the plurality of sensing elements (1) is printed on the at least one substrate (2). This embodiment is highly suitable to create cementitious material structures with sensors in situ and does not need a. previous preparation of the sensing elements (1). Through. different methods that are subject of the present invention, it is quite simple and versatile to create structures with sensors based on the cementitious material, making any structure a potential point of interaction.

Also in this embodiment, and more specifically, the at least one substrate (2) is a substrate of a non-cementitious material, such as a polymeric material, paper, derivatives of paper, ceramic or glass. In case the substrate (2) is polymer, it may consist of polyethylene terephthalate (PET) or polyethylene naphthalate (PEN). The substrate (2) of a non-cementitious material thus consists of materials compatible with roller or sheets systems.

These substrates present characteristics of dimensional stability, levelled surface, temperature resistance, namely till 160° C., and moisture resistance.

Alternatively to a non--cementitious substrate (2), the at least one substrate (2) consists in the cementitious material itself or in a micro concrete layer, and the printing surface of the plurality of sensing elements (1) in the cementitious material forms a substantially finite plane parallel to the cuter surface (4) of the structure. This type of substrate (2) is shaped in a mold for filling with cementitious material (13).

The sensors making part of the plurality of sensing elements (1) may consist in capacitive sensors, inductive sensors, sensors of electro-dermal activity, blood volume pulse, temperature, luminosity, sound, or a combination thereof, among others.

In the case of sensing elements (1) consisting of blood volume pulse (BVP) sensors of optoelectronic technology, the cementitious material structure with sensors additionally includes optical fibers arranged in such a way that one of the ends of each optical fiber is at the outer surface (4) of the structure and the other end is at; the surface of each sensing element (1). This innovative configuration allows the sensing elements (1), which include a light source and a photodetector, to be encapsulated in the cementitious materials while simultaneously measuring BVP signals from the outside of the structure.

Preferably, the blood volume pulse (BVP) sensors of optoelectronic technology are not printed, but are rather prefabricated by one of the methods known in prior art.

In most complex aspect of the present invention, it is intended to obtain a touchpad type interface destined to a human user.

Thus, the present invention includes a configuration of the cementitious material structure with sensors in which the plurality of sensing elements (1) is arranged in a mesh, allowing interaction with the user or the external element through the detection by different sensing elements (1) of the mesh. The mesh configures a plane parallel to the outer surface (4).

In a further possible embodiment of the mesh configuration, the cementitious material structure with sensors includes several planes parallel to each other and to the outer surface, having a plurality of sensing elements (1) and defining a three-dimensional mesh of sensing elements (1).

In an embodiment alternative or additional to the previous one, the plurality of sensing elements (1) of the cementitious material structure with sensors is arranged in such a way that a central sensing element (5) is surrounded by at least two peripheral sensing elements (6), arranged on a line which is part of a plane parallel to the outer surface (4) of the cementitious material structure with sensors.

In an embodiment additional to the previous one, the cementitious material structure with sensors includes at least four peripheral sensing elements (6) arranged in pairs in at least two perpendicular imaginary lines, which are part of a plane parallel to the outer surface (4) of the cementitious materials structure with sensors and intersect at midpoint of the central sensing element (5).

In both embodiments, the cementitious material structure with sensors allows the detection of the movements of the user over the surface with sensors, through individualized sensing elements (1).

In the case of tracking the approach of a detectable element by the sensing elements (1), it is possible to detect the exact position of the change in the sensing elements (1) and therefore detect the position of the element that caused the change.

For the treatment of data from the plurality of sensing elements (1), the present invention includes signal conditioning means (7), at least one microcontroller (8) and optionally local and/or remote communication means (9). The signal conditioning means (7) is connected to the plurality of sensing elements (1), being suitable to convert and adapt the signals from the sensing elements (1) into analogue/digital inputs of the at least one microcontroller (8), which processes the data and sends it via digital/analog communication to a wired/wireless central system.

In order the structure that is object of the present invention to present the information, thus providing said interactivity, the cementitious material structure with sensors further includes, in one embodiment, at least one actuator element (10).

Therefore, the processing of the signals read by the integrated sensors allows generating different outputs in the at least one actuator element (10). This could consist of sound generating means, at least one light emitting diode (LED) or at least a lamp, an output controlling the activation of a device, or a screen displaying the signal read by the sensor, among others.

The analysis of data from the plurality of sensing elements (1) and the consequent actuation on the at least one actuator element (10) may be carried out in situ or remotely, as the local and/or remote communication means (9) is suitable for connecting to a global management control system, which allows a customized interaction with the user.

In the case of the at least one actuator element (10) consists of a LED or another type of light emitting means, and since in the present invention the electronics is integrated inside the concrete, the conduction of the light up to the outer surface (4) is made through a optical fiber which is also integrated into the cementitious material.

The same principle applies in the case in which the sensing elements (1) are, for example, blood volume pulse (BVP) sensors, wherein. each fiber filament contacts, at one end, with the finger of the user, and with the light receiver or emitter located at the other end.

The power supply of the electronic components embedded in the cementitious material structure with sensors consists of finite supply equipment—primary or secondary batteries—or direct connection to the mains, including in this case AC/DC conversion means. The power supply of the electronic elements set which is part of the cementitious material structure of the present invention is performed using a power cable connecting to power means (12), in any of the alternatives listed.

In the case of sensing through printed sensing elements (1) on the at least one substrate (2), these sets are laminated and/or coated with different materials in order to obtain electrical, mechanical and chemical protection after printing and before integration in the concrete structure. As protective materials, waterproof materials and materials with good mechanical and chemical resistance are considered in order to resist the concreting process of the concrete parts. The encapsulation of the printed sensors with these materials can be made through lamination, and/or heat sealing, and/or slot die, and/or doctor blade, and/or knife-over-edge, and/or screen printing, and/or spray, of a polymeric material curable by ultraviolet (UV) light and/or temperature.

It is also an object of the present invention a manufacturing method of the cementitious material structure with sensors which includes the following steps:

a) integrating a plurality of sensing elements (1) on at least one substrate (2);

b) inserting the encapsulated set of the plurality of sensing elements (1) and the at least one substrate (2) inside the formwork;

c) filling with cementitious material.

More specifically, the manufacturing method of the present invention further includes the following step, performed after step a):
encapsulation of the set of the plurality of sensing elements (1) and the at least one substrate (2) with a waterproofing material (3).

Still more specifically, regarding the configuration of cementitious material structure with sensors in which the sensing elements (1) are printed on at least one substrate (2), step a) consists more specifically in printing a plurality of sensing elements (1) on at least one substrate (2).

This step is suitable for all configurations of the cementitious material structure of the present invention, in which the sensing elements (1) are formed in situ, and is not suitable for the cases in which these are prefabricated by means of any methods known in prior art, and just integrated in situ on the at least one substrate (2).

The printing step may consist of one of the following techniques, or combinations thereof:
screen printing;
rotogravure;
ink jet printing in roller or sheets systems.

Encapsulation of the plurality of sensing elements (1) and the at least one substrate (2) may consist of a layer produced by lamination and/or coating with different materials—in order to obtain electrical, mechanical and chemical protection—after printing and before integration in the concrete structure and involvement with cementitious material. As protective materials, waterproof materials and materials with good mechanical and chemical resistance are considered in order to resist the concreting process of the concrete parts. The encapsulation of the printed sensors with these materials can be made through lamination, and/or heat sealing, and/or slot die, and/or doctor blade, and/or knife-over-edge, and/or screen printing, and/or spray, of a polymeric material curable by ultraviolet (UV) light and/or temperature.

In the case of the substrate (2) consists of a non-cementitious substrate, the method includes more specifically the following steps:
phasing the concreting into outer and inner wall concreting;
introduction of the plurality of sensing elements (1) printed on at least one substrate (2), after its encapsulation, into the formwork during the preparation of the inner wall concreting;
in the introduction of previous step, positioning the plurality of sensing elements (1) printed on at least one substrate (2) with the aid of spacers made of cementitious material, and its attachment to the formwork reinforcement by means of spring systems or equivalent, in order to ensure stability and positioning during concreting and to comply with their actuation fields regarding the section of the part to be concreted;
gradual introduction of the cementitious material so as not to damage the sensing systems and accessory parts.

Also in this case, the method more specifically includes the following steps:
use of negatives in the parts, for placement and assembly of connecting boxes at strategic points previously defined in project;
concreting the entire set with finishing concrete or coating concrete, depending on the dimensioning and the planned type of finishing.

The outer face is concreted according to the common procedure.

In the case, alternative to the above, of prefabrication of structures for end use—as is the case of street furniture—the method further includes the following steps of phased concreting by means of layers:
in a first layer the concrete is placed and the set of the plurality of sensing elements (1) and the at least one substrate (2) is inserted under this layer;
subsequently, another layer of concrete is placed.

These parts offer a finishing of the face under sight with architectural effects. Also in this case, negatives will be left in these parts to ensure the placement or accessories.

In a case which can be designated as mixed, wherein the plurality of sensing elements (1) is first incorporated into a prefabricated substrate (2) of cementitious material—such as micro concrete, mortar or compatible material—which encapsulates the sensors protecting them mechanically or against moisture and chemical attack, the method more specifically includes the following steps:
pre-molding the cementitious material and printing the plurality of sensing elements (1), with all its connection points covered;
placing the structure into the construction of the cementitious material structure with sensors—for instance wall, floor, decorative part, or street furniture—prior to its concreting or final coating with the cementitious materials or others;
attachment between the reinforcement and the formwork using support hooks and spacers in the case of elements to be concreted, or by means of gluing to the support in order to receive the final coating in the case of application under a plaster, screed or another coating.

Except for those configurations in which sensing elements (1) consist of BVP sensing elements or other type of sensor whose monitored variable is related to light, the sensing elements (1) are significantly close to the surface of the cementitious material structure with sensors, being covered with cementitious material and being at a depth of at least 0.5 cm.

The present invention further relates to a method of operation of the cementitious material structure with sensors, which includes the following steps:
detection of stimulus external to the cementitious material structure with sensors, through the plurality of sensing elements (1);
conditioning the signals produced by the plurality of sensing elements (1);
processing the signals from the plurality of sensing elements (1) in at least one microcontroller (8);
actuation of the electronic element by the at least one microcontroller (8), wherein the electronic element is at least one actuator element (10) and/or a local and/or remote communication. means (9).

### Embodiments

In an embodiment of the object of the present invention, the waterproofing material (3) is a polymeric material.

In another embodiment of the object of the present invention, the at least one substrate (2) consists of a polymeric substrate.

In one embodiment in which the sensing elements (1) are printed on the at least one substrate (2), each sensing element (1) includes a sensing element extension (11) for connection to the other electronic elements with functions such as signal conditioning or signal processing,

In one embodiment of the object of the present invention, the cementitious material consists of concrete, mortar or micro concrete.

In another embodiment of the present invention, the cementitious material consists of concrete with aesthetically appealing features, through using different colors/textures at least on the outer surface of the structure.

In different embodiments of the present invention, the printing ink includes conductive materials such as silver, carbon, nickel, gold, platinum, polymeric materials such as PEDOT:PSS.

In another embodiment which may be combined with the above, the cementitious material structure with sensors includes optical fibers, allowing light emission at the concrete surface without affecting their surface characteristics and durability. Such optical fibers are inserted during the concreting process, and allow the light emission at the concrete surface without affecting their surface characteristics and durability. These are the only elements placed at the surface of the cementitious material structure with sensors, of course besides the cementitious material itself.

In one embodiment of the present invention, the cementitious material structure with sensors consists of a blood volume pulse (BVP) meter, in which the sensing elements (1) are BVP sensors and the at least one actuator element (10) is a light emitting diode (LED).

In this case, the light emitters and receivers are in direct contact with the surface of the external element, as for example the finger of a user (the extremity of the body where the signal measurement is usually held in conventional systems), the conduction of the light being accomplished using optical fiber as previously described.

The integration of optical fibers is also carried out in order to generate dynamic effects by actuating the sensors through touch or proximity, at the concrete surface.

This integration is performed through the rigorous positioning of the fiber in the contact surface, in order to capture the light reflected by the skin after exposure to the light emitted by the emitting fiber. This positioning is pre-molded and subsequently embedded in the final element, or properly positioned in the formwork before concreting.

In different embodiments of the method of the present invention, the manufacture of the cementitious material structure with sensors may include artistic and architectural finishing, coloring and various textures which make its appearance decorative and pleasing, facilitating their integration in existing structures without damage and adding aesthetic value to them, and giving maximum comfort to users.

In a more specific embodiment relating the case in which the present invention includes a configuration of the cementitious material structure with sensors where the plurality of sensing elements (1) is arranged in such a way that a central sensing element (5) is surrounded by at least two peripheral sensors elements (6), the sensing elements (1) consist of different electrodes of a conductive material, such as silver electrodes printed on rigid or flexible substrates, in which they are arranged according to a given geometry.

These electrodes may be encapsulated to improve its strength and durability and to integrate them into the concrete, or materials can be used—preferably FR-4—wherein the surface is coated. with a conductive material (copper, silver, gold alloys, etc.). In these materials the electrodes are deployed in the geometrical arrangement necessary for the correct spatial signal detection (XYZ).

These electrodes are at least connected to the signal conditioning means (7), which in turn is connected to the at least one microcontroller (8).

In a preferred embodiment of the previous example, the at least one microcontroller (8) is connected to the local and/or remote communication means (9).

In the following, different examples of application of the structure and method of the present invention are presented.

### EXAMPLE 1

### Development of Switches and Buzzers in Concrete

In this example a method is considered with the following characteristics

i) Integration of capacitive printed sensors in concrete, during the concreting process according to the method of the present invention.

The cementitious material structure with sensors specifically includes the following elements:

i) signal conditioning means (7), a microcontroller (8);

ii) at least one actuator element (10) consisting of a sound generating actuator and/or a lighting means actuator;

iii) the sensing elements (1) consist of touch or proximity capacitive sensors in the concrete surface;

iv) the at least one substrate (2) consists of flexible polymeric substrates where the capacitive sensors are printed and may be encapsulated to improve its strength and durability regarding its integration in concrete.

The ink used for printing includes conductive materials such as silver, carbon, nickel, gold or platinum, and polymeric materials such as PEDOT:PSS. As a printing technique for these capacitive sensors on the flexible polymeric substrates, rotogravure, or screen printing, or ink jet printing may be considered.

The substrate (2) consists of a polymeric film based in polyethylene terephthalate (PET). The protection of the sensor is obtained by using coating lamination, wet lamination and dry lamination.

The flexible polymeric substrates with printed sensors are integrated in the concrete during the concreting process.

This integration is performed in a process called bilayer, in which the sensor is integrated between layers, thus constituting a high mechanical strength pre-molded element with sensors, which may later be embedded in walls, floors or other constructive elements (integrated during the concreting or in posterior coatings with mortars or screeds) or simply be directly exposed in similar supports.

### EXAMPLE 2

### Development of a Concrete Floor to Security Warnings

In this example a method is considered with the following characteristics:

i) Integration of printed piezoelectric sensors in concrete, during the concreting process according to the method of the present invention.

The cementitious material structure with sensors specifically includes the following elements:

i) signal conditioning means (7), a microcontroller (8);

ii) at least one actuator element (10) consisting of a sound generating actuator and/or a lighting means actuator;

iii) the sensing elements (1) consist of piezoelectric sensors in the concrete surface;

iv) optical fibers, in order the activation of the lighting means is visible at the concrete surface.

Piezoelectric sensors are identified for integration in the concrete. This type of sensors is encapsulated using epoxy resins, or others enabling the sensor protection against aggression of usage and concreting process.

The integration of the sensing elements (1) in concrete is carried out using the bilayer process of concreting, where the sensing elements (1) are integrated between the two layers.

The activation of the lighting means enables issuing a warning or message at the concrete surface. The control signal, for feedback at the respective concrete slab, can be obtained through the use of wiring (physical interconnection between the concrete slabs) or alternatively via wireless communication, for example via radio frequency (RF), Wi-Fi, Bluetooth, ZigBee, or other type of wireless communications protocol.

In a preferred embodiment, the ZigBee is used. This wireless communication protocol, considered preferred, involves the use of a transceiver integrated on each of the concrete substrates (2), allowing them to communicate with each other.

As an application example, the floor issuing security alerts to warn drivers of a. pedestrian approaching in crossing zones is considered.

In a preferred embodiment the lighting means are integrated in the concrete part, by using optical fibers which conduct the emitted light to the concrete surface, ensuring their characteristics and durability.

This set is remotely connected to a global management system for controlling the sensors remotely and customizing the type of interaction with the user and the way the actuator elements (10) act.

### EXAMPLE 3

### Development of Interactive Games in Concrete

In this example a method is considered with the following characteristics:

i) levelling the surface of the cementitious material—concrete;

ii) printing sensors at the concrete surface;

iii) integration of signal conditioning means (7), a microcontroller (8), and at least one actuator element (10) consisting of a sound generating actuator and/or a lighting means actuator;

iv) integration of optical fibers in the concrete, in order the activation of the lighting means is visible at the concrete surface.

This application includes the development of concrete parts with surface levelled by flattering and mechanical grinding after concreting, followed by sealing based. on silanes-siloxanes and acrylic resins chemically compatible with the printing ink.

The printing of sensors at the concrete surface can be carried out using inks with conductive materials, for example silver, carbon, nickel, gold, platinum, polymeric materials such as PEDOT:PSS.

The printing is performed by rotogravure, or screen printing, or ink jet.

The sensor printed on the concrete surface is protected using waterproofing paints such as acrylic or silicate, or by means of micro concrete, mortar or compatible cementitious coating.

The technology or the integration of printed sensors on polymeric substrates, or the so called traditional sensing of concrete, allows developing interactive demonstrators for use as concrete parts in interactive street furniture, or flooring, or walls. These concrete parts can he applied to interactive games, through touch or proximity action.

### EXAMPLE 4

### Reading the Heartbeat by Contact With the Concrete Surface

In this example a method is considered with the following characteristics:

i) Integration of printed BVP sensors in concrete, during the concreting process according to the method of the present invention.

The cementitious material structure with sensors specifically includes the following elements:

i) signal conditioning means (7), a microcontroller (8) and local and remote communication means (9);

ii) the sensing elements (1) consist of BVP sensors.

The integration of the BVP sensors in concrete is carried out by connecting the fibers ends embedded in the part.

This scheme has several possible applications, among which the most common is the heartbeat measurement through detection of peaks in the photoplethysmogram, but there may also exists applications in the detection of heart rate variability (HRV), or in the evaluation studies of arterial resistance and aorta elasticity.

Other applications of the sensor can be developed, since this allows obtaining vital information. For example, in studies of sleep disorders, the BVP sensors have been used to extract some parameters of arousal during sleep.

All these features, integrated in concrete, make it possible monitoring these parameters—for instance in private homes, nursing homes—by any user without the need for a specialized technician.

The data obtained are processed and monitored by a global management control system, and this data can be viewed remotely. If the values do not, match the normal parameters (read values exceeding reference limits) an alert is sent to a user-defined contact.

As will be apparent to one skilled in the art, the present invention should not be limited to the embodiments described herein, and various changes are possible which remain within the scope of the present invention.

Of course, the preferred embodiments presented above can be combined in different possible ways, being herein avoided the repetition of all such combinations.

The invention should be limited only by the spirit of the following claims.

1. Cementitious material structure with sensors characterized in that it includes at least one substrate (2), a plurality of sensing elements (1) arranged on the at least one substrate (2) and including cementitious material involving the whole set.

2. Cementitious material structure with sensors according to claim 1, characterized in that it further includes a waterproofing material layer (3) covering the set of the plurality of sensing elements (1) and the at least one substrate (2).

3. Structure according to claim 1, characterized in that the plurality of sensing elements (1) is arranged on a surface substantially parallel to an outer surface (4) of the cementitious material structure with sensors.

4. Structure according to claim 1, characterized in that the plurality of sensing elements (1) is a plurality of sensing elements (1) printed on the at least one substrate (2).

5. Structure according to claim 1, characterized in that the at least one substrate (2) consists of a non-cementitious material substrate preferably consisting of a polymeric material, glass, paper, derivatives of paper, or ceramic material,

6. Structure according to claim 3, characterized in that the at least one substrate (2) consists in the cementitious material itself or a micro concrete layer, and the printing surface of the plurality of sensing elements (1) forms in cementitious material a substantially finite plane parallel to the outer surface (4) of the structure.

7. Structure according to claim 4, characterized in that the sensing elements (1) consist of capacitive and inductive sensors, sensors of electro-dermal activity, temperature, luminosity, or sound, being that the sensing elements (1) preferably consist of blood volume pulse sensors, and that the structure further includes optical fibers arranged in such a way that one of the ends of each optical fiber is at the outer surface (4) of the structure and the other end is at the surface of each sensing element (1).

8. Structure according to claim 1, characterized in that the cementitious material is concrete, mortar or shotcrete.

9. Structure according to claim 1, characterized in that it includes several planes parallel to each other and to the outer surface, which in turn includes a plurality of sensing elements (1), defining a three-dimensional mesh of sensing elements (1) wherein, preferably, the plurality of sensing elements (1) is arranged in such a way that a central sensing element (5) is surrounded by at least two peripheral sensing elements (6), with all the sensing elements (1) arranged on a line which is part of a plane parallel to the surface of the cementitious material structure with sensors.

10. Structure according claim 9, characterized in that it includes at least four peripheral sensing elements (6) arranged in pairs in at least two perpendicular lines which are part of a plane parallel to the surface of the cementitious material structure with sensors and intersect, at midpoint of the central sensing element (5).

11. Structure according to claim 1, characterized in that the waterproofing material (3) is a polymer.

12. Structure according to claim 1, characterized in that it includes signal conditioning means (7), at least one microcontroller (8), and optionally local or remote communication. means (9) and/or it includes at least one actuator element (10).

13. Structure according to claim 12, characterized in that it further includes optical fibers arranged in such a way that one of the ends of each optical fiber is at the outer surface (4) of the structure and the other end is at the surface of each actuator element (10), the actuator elements (10) consisting of light generating elements.

14. Method for manufacturing the cementitious material structure with sensors according to claim 1, characterized in that it includes the following steps:
a) integrating a plurality of sensing elements (1) on the at least one substrate (2);
b) inserting the encapsulated set of the plurality of sensing elements (1) and the at least one substrate (2) inside the formwork;
c) filling with cementitious material.

15. Method according to claim 14, characterized in that it further includes the following step, performed after step a):
encapsulation of the set of the plurality of sensing elements (1) and the at least one substrate (2) with a waterproofing material (3) step a) consisting preferably of printing a plurality of sensing elements (1) on at least one substrate (2).

16. Method according to claim 15, characterized in that the printing consists of one of the following techniques, or combinations thereof:
screen printing;
rotogravure;
ink jet printing in roller or sheets systems.

17. Method according to claim 14, characterized in that, in case the substrate (2) consists of a non-cementitious substrate, it includes the following steps:
phasing the concreting into outer and inner wall concreting;
introduction of the plurality of sensing elements (1) printed on at least one substrate (2), after its encapsulation, into the formwork during the preparation of the inner wall concreting;
in the introduction of previous step, positioning the plurality of sensing elements (1) printed on at least one substrate (2) with the aid of spacers made of cementitious material, and its attachment to the formwork reinforcement by means of spring systems or equivalent;
gradual introduction of the cementitious material so as not to damage the sensing elements and accessory parts.
use of negatives in the parts, for placement and assembly of connecting boxes at strategic points previously defined in project;
concreting the entire set with finishing concrete or coating concrete, depending on the dimensioning and the planned type of finishing.

18. Manufacturing method according to claim 14, characterized in that, in case the substrate (2) consists in the cementitious material itself, the concreting is phased by layers of prefabricated structures for final use, including the following steps:
placing the concrete in a first layer and inserting under this layer the set of the plurality of sensing elements (1) and the at least one substrate (2);
placing thereafter another concrete layer.

19. Manufacturing method according to claim 14, characterized in that the plurality of sensing elements (1) is previously incorporated in a prefabricated substrate (2) of cementitious material—such as micro concrete, mortar or compatible material—further including the following steps:
pre-molding the cementitious material and printing the plurality of sensing elements (1), with all its connection points covered;
placing the structure into the construction of the cementitious material structure with sensors—for instance a wall, floor, decorative part, or street furniture—prior to its concreting or final coating with the cementitious materials or others;
attachment between the reinforcement and the formwork using support hooks and spacers in the case of element to be concreted, or by means of gluing to the support in order to receive the final coating in the case of application under a. plaster, screed or another coating.

20. Method for operating the cementitious material structure with sensors according to claim 1, characterized in that it includes the following steps:
detection of stimulus external to the cementitious material structure with sensors, through the plurality of sensing elements (1);
conditioning the signals produced by the plurality of sensing elements (1);
processing the signals from the plurality of sensing elements (1) in at least one microcontroller (8);
actuation of the electronic element by the at least one microcontroller (8), wherein the electronic element is at least one actuator element (10) and/or a local or remote communication means (9).
